(19) United States
(12) Patent Application Publication
(10) Pub. No.: US 2005/0154285 A1
(43) Pub. Date: Jul. 14, 2005

(54) System and method for receiving and displaying information pertaining to a patient

(76) Inventor: Curtis G. Neason, New York, NY (US)

Correspondence Address: GE MEDICAL SYSTEM;C/O FOLEY & LARDNER, 777 EAST WISCONSIN AVENUE, MILWAUKEE, WI, 53202-5367, US

(21) Appl. No.: 10/751,089

(22) Filed: Jan. 2, 2004

(51) Int. Cl. A61B 5/05

(52) U.S. Cl. 600/407

(57) ABSTRACT

The present description relates to a system comprising an electrophysiology module and a patient monitoring module. The electrophysiology module is configured to receive electrical information pertaining to a heart. The electrical information is sensed using a probe positioned inside the heart. The electrophysiology module is also configured to receive position information pertaining to a position of the probe. The patient monitoring module is communicatively coupled to the electrophysiology module. The patient monitoring module is configured to receive at least two of the following types of patient information: blood pressure, temperature, respiratory rate, pulse oximetry, and respiratory CO₂ concentration.

### BACKGROUND

The present description relates generally to systems and methods for receiving and displaying various patient information. In particular, the present description relates to systems and methods for receiving and displaying information pertaining to a patient in the context of an electrophysiology (EP) study.

EP studies can be used to diagnose and treat a number of serious heart problems. One type of heart problem that can be diagnosed and treated by conducting an EP study is a cardiac arrhythmia. A cardiac arrhythmia can generally be referred to as an abnormal heart rhythm such as tachycardia, bradycardia, etc. One particularly dangerous arrhythmia that is often diagnosed and treated using an EP study is ventricular fibrillation. Left untreated, an arrhythmia presents a serious health risk to an individual.

In a typical EP study, a catheter is inserted into a vein or artery (e.g., in the groin, etc.) and guided to the interior of the heart. Once inside the heart, the catheter is contacted with the endocardium at multiple locations. At each location, the position of the catheter and the electrical properties of the endocardium can be measured. The attending physician uses this information to assist in locating the origin of a cardiac arrhythmia. The results of the EP study may lead to further treatment, such as ablating the area of the heart causing the arrhythmia, implanting a pacemaker or cardioverter defibrillator, or prescribing medication to treat the arrhythmia. Generally, ablating an area of the heart renders it electrically inoperative thus removing stray impulses and restoring the heart's normal electrical activity.

In a typical EP study where the system used for the EP study includes structural mapping capabilities, the patient's vital signs are monitored and recorded by a vital signs monitoring system that is separate and not in communication with the system used to conduct the EP study. Monitoring the patient's vitals allows the attending physician or physicians to know if there are any potential problems as the EP study proceeds. In a typical situation, the patient's vital signs are recorded periodically as the procedure proceeds. This may be done by a nurse who reads the vital signs from the vital signs monitoring system or the vital signs monitoring system may be configured to periodically record the patient's vitals. In some instances, the vital signs monitoring system may be configured to print out the patient's vitals.

Unfortunately, having two separate systems connected to the patient is often undesirable. For example, there may be some overlap in the type of information acquired by the EP system and the vitals monitoring system. This may result in additional duplicative cabling which may increase the potential for interference between the extra cables. Also, the cost of having two systems may be greater because of the extra cabling, training personnel on both systems, etc. Accordingly, it would be desirable to provide an improved system and method for acquiring and displaying EP information and/or structural mapping information in conjunction with the vital signs of the patient.

Of course, the claims define the scope of the subject matter for which protection is sought, regardless of whether any of the aforementioned disadvantages are overcome by the subject matter recited in the claims. Also, the terms recited in the claims should be given their ordinary and customary meaning as would be recognized by those of skill in the art, except, to the extent a term is used herein in a manner more expansive than its ordinary and customary meaning, the term should be given its ordinary and customary meaning plus the additional expansive meaning, or except if a term has been explicitly defined to have a different meaning by reciting the term followed by the phase “as used herein shall mean” or similar language. Accordingly, the claims are not tied to any particular embodiment, feature, or combination of features other than those explicitly recited in the claims.

### SUMMARY

One embodiment relates to a system comprising an electrophysiology module and a patient monitoring module. The electrophysiology module is configured to receive electrical information pertaining to a heart. The electrical information is sensed using a probe positioned inside the heart. The electrophysiology module is also configured to receive position information pertaining to a position of the probe. The patient monitoring module is communicatively coupled to the electrophysiology module. The patient monitoring module is configured to receive at least two of the following types of patient information: blood pressure, temperature, respiratory rate, pulse oximetry, and respiratory CO₂ concentration.

Another embodiment relates to a system comprising a probe configured to be positioned inside a heart of a patient and a console comprising computer components which are communicatively coupled together and configured to receive the electrical information from the probe. The probe is configured to sense electrical information pertaining to the heart. The computer components are also configured to receive position information pertaining to one or more positions of the probe and patient information which comprises at least two of the following types of information: blood pressure, temperature, respiratory rate, pulse oximetry, and respiratory CO₂ concentration.

Another embodiment relates to a system comprising one or more processors communicatively coupled together and configured to receive electrical information, position information, and patient information. The electrical information pertains to a heart, the electrical information is sensed using a probe positioned inside the heart. The position information pertains to a position of the probe. The patient information comprises at least two of the following types of information: blood pressure, temperature, respiratory rate, pulse oximetry, and respiratory CO₂ concentration.

Another embodiment relates to a system comprising one or more processors communicatively coupled together and configured to receive electrical information, position information, and patient information. The electrical information pertains to a heart, the electrical information is sensed using a probe positioned inside the heart. The position information pertains to a position of the probe. The patient information comprises at least two of the following types of information pertaining to the patient: blood pressure, temperature, respiratory rate, pulse oximetry, and respiratory CO₂ concentration. The system is configured to generate a report comprising the patient information and at least one of the electrical information and the position information.

### BRIEF DESCRIPTION OF THE DRAWINGS

FIG. 1 is a system for receiving and displaying patient information according to one embodiment.

FIG. 2 is a display for displaying patient information according to one embodiment.

FIGS. 3 and 4 are various embodiments of systems for receiving and displaying patient information.

### DETAILED DESCRIPTION

The present description is generally provided in the context of a system which is configured to receive and display patient information (e.g., electrical information pertaining to the heart, structural information pertaining to the heart, vitals information, etc.). Although, the present description is provided primarily in the context of receiving and displaying vitals information pertaining to a patient along with electrical and/or structural information of the heart, it should be understood that the systems and methods described and claimed herein may also be used in other contexts as would be recognized by those of ordinary skill. It should also be understood that a particular example or embodiment described herein may be combined with one or more other examples or embodiments also described herein to form various additional embodiments as would be recognized by those of ordinary skill. Accordingly, the systems and methods described herein may encompass various embodiments and permutations as may be desired.

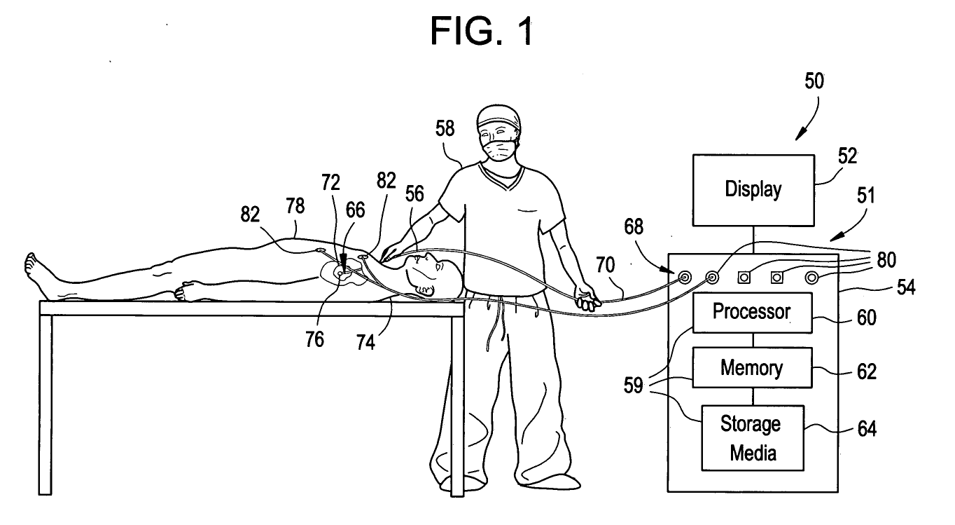

Referring to FIG. 1, one embodiment of a system 50 is shown. System 50 includes a console or computer 51 and a probe 56. System 50, broadly described, may be used to receive and display various types of patient information. In particular, system 50 may be used to simultaneously and/or selectively receive and/or display electrical and/or structural information pertaining to a heart 72 and vitals information pertaining to a patient 74.

System 50 may be a wide variety of systems used for an equally wide variety of uses. For example, in one embodiment, system 50 may be any system that is configured to use one or more probes 56 inside the body to measure, monitor, diagnose, manipulate, and/or otherwise provide information about heart 72. In another embodiment, system 50 may be an EP monitoring and diagnostic system that is configured to use probe 56 to purposefully alter and/or provide information regarding the electrical activity of heart 72. In another embodiment, system 50 may be an EP monitoring and diagnostic system that is configured to measure one or more positions of probe 56, which may be used to create a structural map of heart 72. In another embodiment, system 50 may be a combination of the previous embodiments. Thus, system 50 may be configured to receive and/or display electrical and/or structural information pertaining to heart 72 as well as be configured to receive and/or display vitals information pertaining to patient 74.

As shown in FIG. 1, probe 56 and display 52 are communicatively coupled to computer components 59 in cabinet 54. Information sensed by probe 56 may be communicated to computer components 59. Information from computer components 59 may then be communicated to display 52 where it is displayed to a nearby person 58 (e.g., attending physician, nurse, technician, etc.). The configuration shown in FIG. 1 is only one of many suitable configurations. For example, in another embodiment, probe 56 may be communicatively coupled directly to display 52. In this embodiment, display 52 may be configured to display the information provided by probe 56 without the information being communicated through cabinet 54 (e.g., display 52 comprises the necessary computer components 59 to receive information from probe 56). In another embodiment, display 52 may be combined with cabinet 54 so that the functions generally performed by computer components 59 in cabinet 54 and display 52 are performed by the combined unit (e.g., display 52 comprises all of computer components 59). In another embodiment, console 51 may include two or more displays 52. For example, one display may be used to display electrical and/or structural information pertaining to heart 72 and the other display may be configured to display the vitals information. Of course, a wide variety of information may be displayed on display 52. In one embodiment, display 52 may be configured to be at a position that is convenient for person 58 to view (e.g., display 52 is positioned at eye level of person 58 when person 58 is standing, etc.) as person 58 moves probe 56.

System 50 may be configured to receive and/or display various information pertaining to patient 74. For example, in one embodiment, system 50 may be configured to receive and/or display vitals information pertaining to patient 74. Vitals information may include one or more, in any combination, of the following types of patient information: electrocardiogram (ECG), pulse oximetry (SpO₂), non-invasive blood pressure (NIBP), temperature, respiratory rate, respiratory CO₂ concentration (etCO₂), impedance cardiography (ICG), pulse rate, cardiac output (CO), etc. System 50 may also include sensors that are coupled to computer components 59 in console 51 to provide this information. In one embodiment, display 52 may be configured to display at least one, two, three, four, or all five of the following types of information pertaining to patient 74: blood pressure, temperature, respiratory rate, pulse oximetry, respiratory CO₂ concentration, and pulse rate.

System 50 may be configured to include additional components and systems. For example, system 50 may comprise a printer. The printer may be configured to print on standard sized pages or may be configured to print on smaller rolls of paper. The printer may also be used to periodically or continuously print out vitals information for patient 74. The printer may also be used to print out a report at the end of an EP study. System 50 may also be configured as part of a network of computers (e.g., wireless, cabled, secure network, etc.) or as a stand-alone system. Information pertaining to patient 74 may be transmitted over the network and stored as part of a data record for patient 74.

Computer components 59 in cabinet 54, shown in FIG. 1, comprise a processor 60, memory 62, storage media 64, and one or more input devices (e.g., mouse, keyboard, etc.). Computer components 59 are configured to receive information from probe 56, process the information, and provide output using display 52. The information provided to computer components 59 may be continually stored (i.e., all information is stored as it is received) or intermittently stored (i.e., periodic samples of the information are stored) using storage media 64 (e.g., optical storage disk such as a CD, DVD, etc., high performance magneto optical disk, magnetic disk, etc.). In general, storage media 64 differs from memory 62 in that storage media 64 is configured to maintain the information even when storage media 64 is not provided with power. In contrast, memory 62 typically does not maintain the information when the power is off.

In one embodiment, console 51 is a desktop computer. In another embodiment, console 51 may include input receivers 80 on cabinet 54 or display 52 that are configured to receive additional information pertaining to patient 74. For example, in one embodiment, input receivers 80 may include one or more input receivers configured to receive input from leads 82 (e.g., ECG leads, etc.). In other embodiments, input receivers 80 may include suitable receivers for receiving vitals information. For example, input receivers 80 may be configured to be coupled to a traditional NIBP arm cuff sensor.

Probe 56 comprises a distal end 66, a proximal end 68, and a probe body 70. In general, probe 56 may be positioned in or adjacent to heart 72 (shown in FIG. 1 in a cross-sectional view to expose distal end 66 of probe 56) of patient 74. In one embodiment, distal end 66 may include one or more sensors 76, which are configured to sense various electrical information (e.g., electrical potential at one or more positions of the endocardium, activation times, etc.) pertaining to heart 72. The electrical information may then be communicated back to console 51 and displayed on display 52. In one embodiment, probe 56 may comprise a plurality of sensors configured to sense the electrical information pertaining to heart 72 (e.g., probe 56 is a balloon or sock catheter, etc.). The electrical information may be used to create an electrical map (e.g., map of the activation times, electrical potentials, etc.) of heart 72.

Probe 56 may be any number of suitable probes having a variety of configurations. For example, probe 56 may include a lumen in which wires may be placed to communicate information from sensors 76 back to console 51 and to transmit an ablation charge from console 51 to distal end 66 to correct the electrical pathways in heart 72. Of course, the lumen may also be used to allow fluid to flow through probe 56.

In another embodiment, a localization system, included as part of system 50, may be used to determine the spatial location of one or more portions (e.g., sensors 76, etc.) of distal end 66 of probe 56. This may be useful in moving probe 56 back to an earlier position or to create a structural map of heart 72. Any suitable localization system may be used as would be recognized by those of ordinary skill. For example, the position of distal end 66 of probe 56 may be determined using one or more transmitters and/or receivers that are located outside the body of patient 74 (typically at least three transmitters and/or receivers are used). In this example, the transmitters and/or receivers may be configured to send and/or receive signals to and/or from distal end 66. These signals may be used to determine the position of distal end 66. In one embodiment, the transmitters and/or receivers may be incorporated into one or more leads 82 positioned on skin surface 78 of patient 74. In another embodiment, the transmitters and/or receivers may be positioned so as not to be in contact with patient 74. In another embodiment, leads 82 may be used to determine the position of distal end 66 of probe 56 by sending a signal that is useful in determining the impedance of probe 56, which may be used to determine the position of probe 56. In another embodiment, the localization system may be configured to determine the position of multiple sensors 76 on distal end 66 of probe 56.

Display 52, shown in FIG. 1, is configured to provide output to a user in the form of information, which may include alphanumeric (e.g., text, numbers, etc.) output, graphical image output, etc. In one embodiment, display 52 may be configured to also receive input from a user (e.g., touch screen, buttons located adjacent to the screen portion of display 52, etc.). Display 52 may be any number of suitable displays in any number of suitable configurations. For example, display 52 may be a liquid crystal display, flat screen display, SVGA display, VGA display, etc.

In one embodiment, display 52 may be configured to display one or more images (computed tomography, magnetic resonance, ultrasound, etc.) of heart 72. Display 52 may also be configured to display a structural and/or electrical map of heart 72. In another embodiment, display 52 may be configured to display vitals information pertaining to patient 74.

Display 52 may also be configured to display one or more representations of one or more probes 56 and the information provided by probes 56. For example, in one embodiment, display 52 may be configured to display a representation of probe 56. In another embodiment, display 52 may be configured to display representations of sensors 76 which are on probe 56. In another embodiment, display 52 may be configured to display the electrical information pertaining to heart 72, which is received from sensors 76 (e.g., a contour map of the electrical properties of heart 72). In another embodiment, display 52 may be configured to display markers showing one or more locations where the electrical information has been sensed. In one embodiment, each marker may display an abbreviated amount of information regarding the electrical information. When a user selects one of the markers, the user is shown a greater amount of electrical information for that particular location of heart 72. In embodiments where the organ or structure comprises heart 72, these markers may be color coded based on the activation times at the various locations inside heart 72 (e.g., red is for early activation times and blue is for late activation times). By displaying a number of markers on display 52, the user can readily observe the electrical information pertaining to various areas of heart 72. Any suitable marker or identifier may be used to represent probe 56 on display 52. For example, in one embodiment, probe 56 may be displayed as a line with a series of points corresponding to sensors 76. The line segments connecting the points represent the portion of probe 56 where there are no sensors. Probe 56 may be shown or represented on display 52 in any of a number of other suitable ways as well.

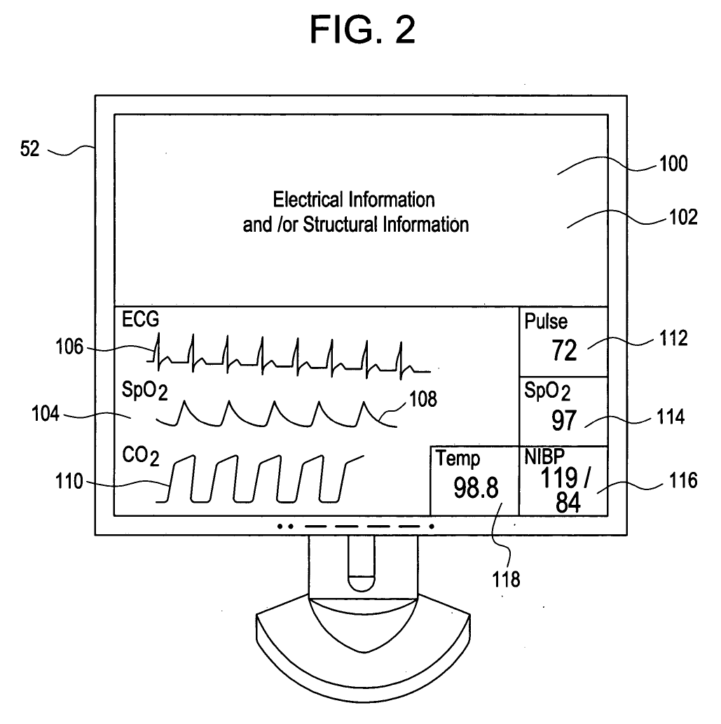

Referring to FIG. 2, one embodiment of display 52 is shown. In this embodiment, display 52 is a flat screen LCD display. Display 52 comprises a screen 100 where information is displayed to a user. In the embodiment shown in FIG. 2, screen 100 is horizontally split in the center. A top portion 102 of screen 100 is configured to display electrical and/or structural information pertaining to heart 72 (e.g., electrical and/or structural maps of heart 72, etc.). Also, top portion 102 may be used to display representations of probe 56 to create an electrical and/or structural map of heart 72, for example.

A bottom portion 104 of screen 100 may be configured to display vitals information pertaining to patient 74. As shown in FIG. 2, vitals information may include an ECG waveform 106, a pulse oximetry waveform 108, and respiratory CO₂ waveform 110. In addition to waveform representations, the vitals information may also be displayed as numerical values. For example, in FIG. 2, numerical values displayed on screen 100 may include pulse rate 112, pulse oximetry 114 (either in conjunction with the waveform of the pulse oximetry or alone), blood pressure 116, and/or temperature 118 of patient 74.

FIG. 2 should be considered as only one embodiment of numerous embodiments. Accordingly, the format for displaying information and the particular types of information displayed may be altered in numerous ways. For example, FIG. 2 only shows one display 52. However, in another embodiments, multiple displays 52 may be used to display the electrical and/or structural information pertaining to heart 72 and the vitals information. In other embodiments, screen 100 may be divided vertically or the vitals information may be configured to be in the background unless it is selected at which time the vitals information is displayed for a predetermined period of time.

In another embodiment, the vitals information may comprise alarms. The alarms may be used to notify the attending staff that a threshold has been breached for a particular type of vitals information. For example, one alarm may be set to activate when the pulse exceeds 100 beats per minute. When an alarm activates a variety of audible and visual signals may be used to notify the appropriate personnel. For example, a light may begin to blink and/or the display may highlight the parameter that has exceeded the threshold. Numerous other signals may also be used when an alarm is activated.

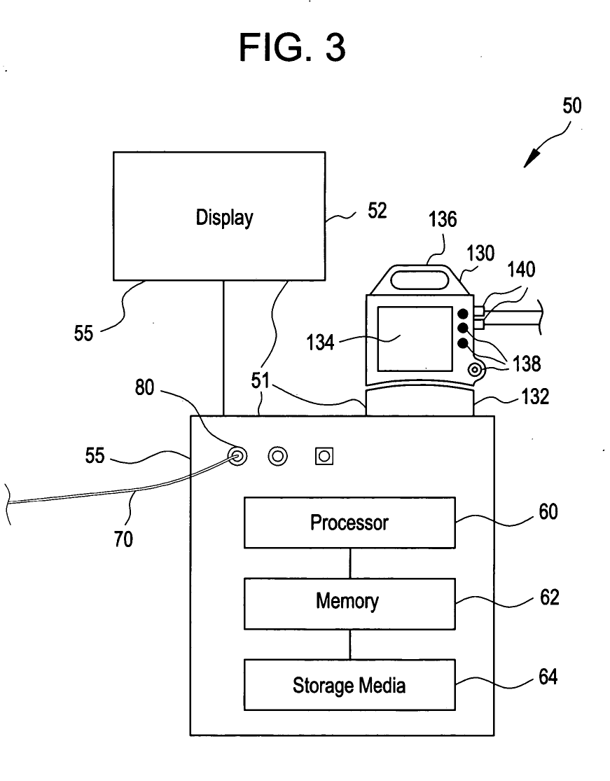

Referring to FIG. 3, another embodiment of system 50 is shown. System 50 includes console 51 which in this embodiment comprises an electrophysiology module (EP module) 55 and a patient monitoring module or vitals monitor 130. EP module comprises computer components 59 and display 52. Vitals monitor 130 comprises computer components which may be similar to computer components 59 (e.g., a processor, memory, inputs, etc.). Vitals monitor 130 is coupled to EP module 55 using docking station 132. Vitals monitor 130 is typically configured to receive and/or display vitals information. Vitals monitor 130 comprises a display 134, handle 136, user input 138, and patient inputs 140. Display 134 is used to display vitals information. In one embodiment, display 134 may be configured to display vitals information in a manner similar to that shown with respect to FIG. 2. For example, display 134 may be configured to display numerical and/or waveforms of various types of vitals information. User inputs 138 are typically buttons, knobs, dials, etc. that allow a user to perform simple tasks such as silence an alarm, switch to various views of display 134, etc. Typically, patient inputs 140 are receivers that are used to acquire the vitals information of patient 74 (e.g., the receivers may be for ECG leads, sensors for an arm cuff to measure blood pressure, etc.). Handle 136 may be used to easily transport vitals monitor 130 from room to room.

In system 50, vitals monitor 130 is configured to be easily removed from EP module 55. In a typical hospital situation, vitals monitor 130 is coupled to patient 74 for much of the time before and/or after a procedure such as an EP study. When patient 74 is moved from room to room during the process of preparing for, performing, and wrapping up the EP study, vitals monitor 130 often accompanies patient 74 in all of these moves. Vitals monitor 130 may be coupled to EP module 55 when patient enters the room where the EP study occurs. In this manner, all of the vitals information available to vitals monitor 130 may be made available to EP module 55 including computer components 59, display 52, and, in general, to the system that is used to acquire electrical and/or structural information pertaining to heart 72. Also, redundant cables, sensors, and inputs are reduced because the vitals information may be communicated from vitals monitor 130 to EP module 55 (e.g., ECG information may be communicated from vitals monitor 130 to EP module 55). Once the procedure is over, vitals monitor 130 may be decoupled from EP module 55 and transported with patient 74 to a recovery room.

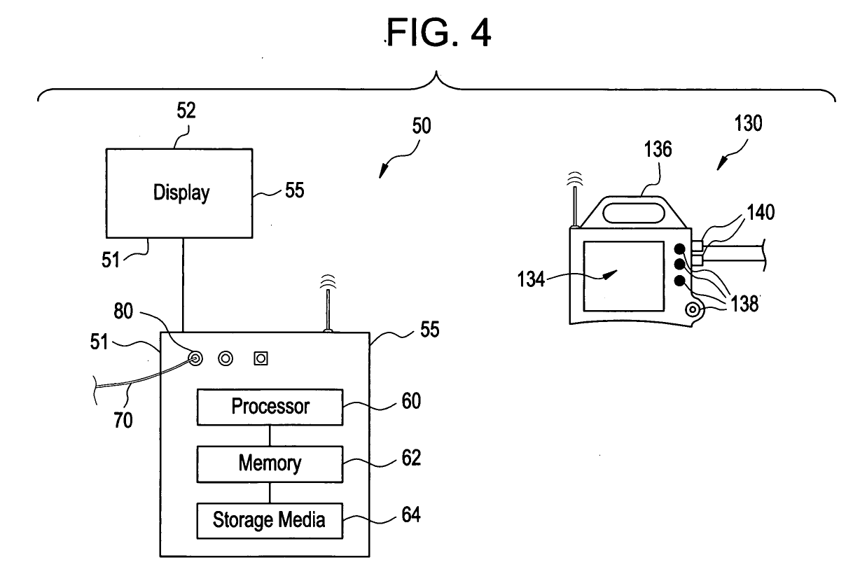

Referring to FIG. 4, another embodiment of system 50 is shown. System 50 comprises vitals monitor 130 and console 51 which includes EP module 55. In this embodiment, vitals monitor 130 is configured to communicate wirelessly with EP module 55. Thus, when patient 74 is brought into the operating room, vitals monitor 130 and EP module 55 establish a communication link. As the EP study is performed, vitals information from vitals monitor 130 may be combined, shared, and/or coordinated with electrical and/or structural information pertaining to heart 72 that is input into EP module 55.

Vitals information may be used to generate a report comprising the vitals information and one or both of the electrical and structural information pertaining to heart 72. In one embodiment the report may comprise patient information such as the name of the physician that performs the EP procedure, the name of the nurse that is present, medications patient 74 may be taking, allergies, history, and/or a description of the procedure. The description of the procedure may provide information about probe 56 (e.g., type of probe, location where probe 56 is inserted into the body, etc.). The report may also include electrical information pertaining to heart 72. For example, the report may include information resulting from pacing heart 72 (e.g., site where pacing was induced, etc.) and/or information about any induced arrhythmias and, in particular, ventricular tachycardia. The report may also include information pertaining to a structural map of heart 72. For example, the report may include information such as the location of probe 56 as it is moved around inside heart 72. The report may also include information pertaining to treatments performed during the procedure. For example, the report may include information about the location and time of an ablation. All of this information may be provided to the physician in an easy to read and understand manner. The report may be especially useful later when examining the patient's 74 medical history to determine any problems or history of illness associated with patient 74.

The vitals information included as part of the report may also have a number of different formats and include widely varying information (e.g., blood pressure, ECG, pulse rate, etc.). For example, the vitals information may be coordinated with the electrical and/or structural information pertaining to heart 72. In one embodiment, selected vitals information may be acquired at the same time as the electrical and/or structural information is acquired. The physician may then refer to the vitals information acquired at the same time as the electrical and/or structural information to explain an unusual reading or pattern. The vitals information may provide additional insight into the electrical and/or structural information.

The following is one embodiment of a report comprising vitals information and electrical and/or structural information. The information provided in the report is only meant to show various types of information that may be used in a particular field, cell, or location and is not meant to represent actual data obtained from a patient.

Referring Physician: Referring Physician, MD
Primary Care Physician: Attending Physician, MD
Nurse: Attending Nurse
Tech: Technician
Current Medications: None
Allergies: None
History: 40 year old male with Hepatitis C and ex-IV drug abuse with
known WPW since age 17. He has had infrequent palpitations in the past
but recently had an episode of prolonged palpitations and was evaluated
for ablation.
Procedure:
After informed written consent was obtained the patient was transported to
the electrophysiology laboratory in the post absorptive, non-sedated state.
The patient was prepped and draped in the usual sterile manner. A 1%
Lidocaine solution was used for local anesthesia. A combination of
Fentanyl, Droperidol and Morphine were used for conscious sedation
throughout the procedure. The patient was continuously monitored
throughout the case per hospital standards. The following sheaths were
placed, after local anesthesia, using the Seldinger technique. In addition
the following electrode catheters were placed under fluoroscopic guidance.

| Site | Sheath | Catheter | Location | Location | Catheter | Location |
|---|---|---|---|---|---|---|
| 1 | 5 F Cordis | | HRA | HRA | | HRA |
| 2 | 6 F Cordis | | RVA | RVA | | RVA |
| 3 | 7 F Cordis | | RVOT | RVOT | | RVOT |
| 4 | 8 F Cordis | | CS | CS | | CS |
| 5 | 6.5 F | | RA | RA | | RA |
| | Locking | | | | | |
| 6 | 10 F Duo | | Tricupid | Tricupid | | Tricupid |
| | | | Ann | Ann | | Ann |
| 7 | 11 F Duo | | LA | LA | | LA |
| 8 | 11 F Trio | | LV | LV | | LV |

After baseline conduction intervals were recorded, programmed extra-
stimulation was performed. Atrial overdrive pacing and extra-stimulation
was performed from the HRA. Ventricular overdrive pacing and extra-
stimulation was performed with up to three extra-stimuli from the LV.
Following intravenous administration of Procainimide programmed
stimulation was repeated. At the end of the procedure the catheters and
sheaths were removed and hemostasis was achieved with pressure. The
patient was transported back to the recovery room in good condition.

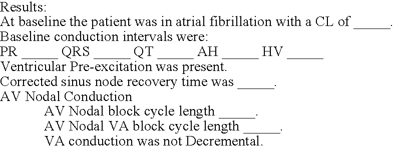

| | | | |
|---|---|---|---|
| Pacing Site | Refractory Site | Drive | ERP |

| | |
|---|---|
| HRA | Atrium |
| RVA | AV Node |
| RVOT | Ventricle |
| CS | Atrium |
| LA | AV Node |
| LV | Ventricle |

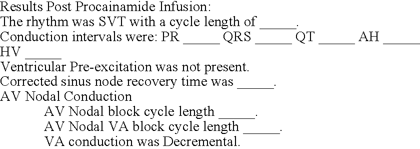

| | | | |
|---|---|---|---|
| Pacing Site | Refractory Site | Drive | ERP |

| | |
|---|---|
| HRA | Atrium |
| RVA | AV Node |
| RVOT | Ventricle |
| CS | Atrium |
| LA | AV Node |
| LV | Ventricle |

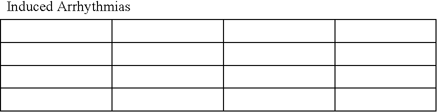

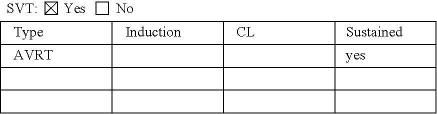

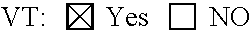

| Induction | CL | Morphology | Sustained | Termination |
|---|---|---|---|---|
| | | Right Bundle Superior Axis | yes | spontanteous |
| | | Right Bundle Inferior Axis | no | burst pacing |
| | | Left Bundle Superior Axis | yes | cardioversion |
| | | Left Bundle Inferior Axis | no | medication |

Mapping:
After the baseline study was completed extensive endocardial mapping
was performed.

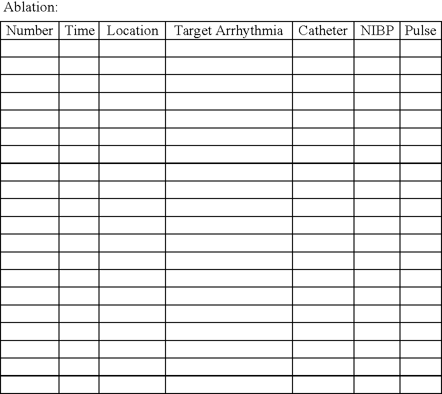

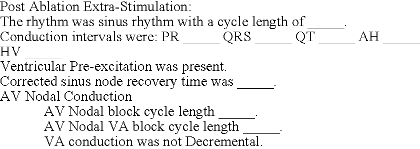

| | | | |
|---|---|---|---|
| Pacing Site | Refractory Site | Drive | ERP |

| | |
|---|---|
| HRA | Atrium |
| RVA | AV Node |
| RVOT | Ventricle |
| CS | Atrium |
| LA | AV Node |
| LV | Ventricle |

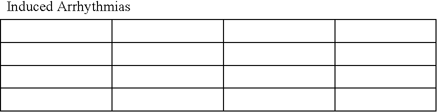

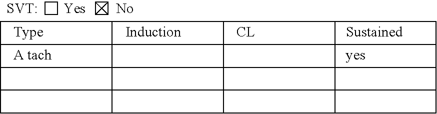

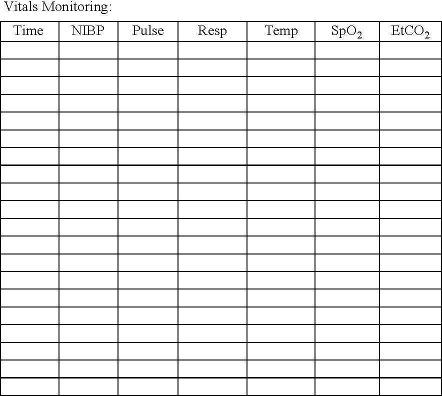

Findings:
1.
2.
3.
Plan:
1.
2.
3.

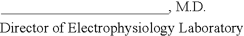

The report shown above is only one example of a suitable report. Accordingly, numerous alterations may be made to the format of the information and what information is included. For example, in one embodiment, the report may include at least two, three, or four of the following types of information pertaining to patient 74: blood pressure, temperature, respiratory rate, pulse oximetry, respiratory CO₂ concentration, pulse rate, and cardiac output. In another embodiment, the report may include graphs of various vitals information recorded during the procedure (e.g., graph of blood pressure, pulse, etc.). In another embodiment, the report may include a map of the electrical properties of heart 72. In another embodiment, the report may include a map of the structure of heart 72 acquired by measuring multiple locations of probe 56.

The construction and arrangement of the elements described herein are illustrative only. Although only a few embodiments have been described in detail in this disclosure, those of ordinary skill who review this disclosure will readily appreciate that many modifications are possible without departing from the spirit of the subject matter disclosed herein. Accordingly, all such modifications are intended to be included within the scope of the methods and systems described herein. The order or sequence of any process or method steps may be varied or re-sequenced according to alternative embodiments. Other substitutions, modifications, changes and omissions may be made in the design, operating conditions and arrangement of the embodiments without departing from the spirit and scope of the methods and systems described herein.

1. A system comprising:
an electrophysiology module configured to receive electrical information pertaining to a heart, the electrical information being sensed using a probe positioned inside the heart, the electrophysiology module also being configured to receive position information pertaining to a position of the probe; and
a patient monitoring module communicatively coupled to the electrophysiology module, the patient monitoring module being configured to receive at least two of the following types of patient information: blood pressure, temperature, respiratory rate, pulse oximetry, and respiratory CO₂ concentration.

2. The system of claim 1, wherein the patient monitoring module is configured to be selectively coupled to and decoupled from the electrophysiology module.

3. The system of claim 1, wherein the patient monitoring module comprises a receiver configured to be coupled to a plurality of sensors used to measure the received patient information.

4. The system of claim 1, wherein the probe is coupled to the electrophysiology module.

5. The system of claim 1, wherein the patient monitoring module and the electrophysiology module are configured to communicate wirelessly with each other.

6. The system of claim 1, wherein the patient monitoring module is configured to receive at least four of the following types of patient information: blood pressure, temperature, respiratory rate, pulse oximetry, and respiratory CO₂ concentration.

7. The system of claim 1, wherein the electrophysiology module comprises a localization system configured to determine the position of the probe.

8. A system comprising:
a probe configured to be positioned inside a heart of a patient, the probe being configured to sense electrical information pertaining to the heart;
a console comprising computer components which are communicatively coupled together and configured to receive the electrical information from the probe, the computer components also being configured to receive position information pertaining to one or more positions of the probe and patient information which comprises at least two of the following types of information: blood pressure, temperature, respiratory rate, pulse oximetry, and respiratory CO₂ concentration.

9. The system of claim 8, wherein the console comprises a receiver configured to be coupled to a plurality of sensors used to measure the received patient information.

10. The system of claim 8, wherein the probe is used to sense activation times of the heart at a plurality of locations on the inside of the heart.

11. The system of claim 10, wherein the position information comprises the position of the probe at the plurality of locations on the inside of the heart where the activation times are sensed.

12. The system of claim 8, wherein the cabinet is configured to receive at least four of the following types of patient information: blood pressure, temperature, respiratory rate, pulse oximetry, and respiratory CO₂ concentration.

13. A system comprising:
one or more processors communicatively coupled together and configured to receive:
electrical information pertaining to a heart, the electrical information being sensed using a probe positioned inside the heart;
position information pertaining to a position of the probe; and
patient information comprising at least two of the following types of information; blood pressure, temperature, respiratory rate, pulse oximetry, and respiratory CO₂ concentration.

14. The system of claim 13, wherein the probe is communicatively coupled to the processor.

15. The system of claim 13, wherein the patient information comprises at least four of the following types of information: blood pressure, temperature, respiratory rate, pulse oximetry, and respiratory CO₂ concentration.

16. The system of claim 13, wherein the probe is used to sense electrical information at a plurality of locations inside the heart, and wherein the position information comprises the position of the probe at the plurality of locations inside the heart.

17. A system comprising:
one or more processors communicatively coupled together and configured to receive:
electrical information pertaining to a heart, the electrical information being sensed using a probe positioned inside the heart;
position information pertaining to a position of the probe; and
patient information comprising at least two of the following types of information pertaining to the patient: blood pressure, temperature, respiratory rate, pulse oximetry, and respiratory CO₂ concentration; and
wherein the system is configured to generate a report comprising the patient information and at least one of the electrical information and the position information.

18. The system of claim 17, wherein the probe is used to sense electrical information at a plurality of locations inside the heart, and wherein the position information comprises the position of the probe at the plurality of locations inside the heart.

19. The system of claim 17, wherein the report comprises an electrical map of the heart created using the electrical information.

20. The system of claim 17, wherein the report comprises a structural map of the heart created using the position information.

21. The system of claim 17, wherein the patient information comprises at least four of the following types of information pertaining to the patient: blood pressure, temperature, respiratory rate, pulse oximetry, and respiratory CO₂ concentration.
